(19) United States
(12) Patent Application Publication
(10) Pub. No.: US 2009/0232949 A1
(43) Pub. Date: Sep. 17, 2009

(54) FLUORESCENT DYE TRACER METHOD FOR ANIMAL FEED SUPPLEMENT PRODUCTS

(75) Inventor: Peter Stark, Cottage Grove, MN (US)

Correspondence Address: MCKEE, VOORHEES & SEASE, P.L.C., 801 GRAND AVENUE, SUITE 3200, DES MOINES, IA, 50309-2721, US

(73) Assignee: ZINPRO CORPORATION, EDEN PRAIRIE, MN (US)

(21) Appl. No.: 12/047,085

(22) Filed: Mar. 12, 2008

(51) Int. Cl. G01N 33/02; A23K 1/00; A23K 1/16; A23K 1/175

(52) U.S. Cl. 426/231; 426/648

(57) ABSTRACT

Compositions for detecting and measuring quantitatively amounts of admix feed additives added to animal feed mixes, using soluble tracer fluorophors, mixed with a liquid form of the additive during the manufacturing process.

### FIELD OF THE INVENTION

The present invention relates to a method of quantitative detection of levels of feed supplement added to animal feeds. The invention also relates to compositions of premix feed supplement that contain homogenous amounts of solubilized fluorescent tracers which are thereafter dried to allow for quantitative detection of the additive.

### BACKGROUND OF THE INVENTION

There has long been a need to identify quantitatively the amount of additives or supplement in animal feed. There are the apparent characteristics that all tracers use to track additives must meet, such as the tracer must be safe; it must be homogeneous; it must be readily detected at low levels, etc. There have been a number of attempts to accomplish this. For example U.S. Pat. No. 6,200,610 describes a coded tracer for animal feeds. U.S. Pat. No. 4,029,820 describes the addition of iron particles that have a dye/indicator coated onto the iron particle. The iron particle can be removed by the use of a magnet and then the indicator developed. These approaches work extremely well to determine if the feed additive is present (qualitative detection) but it suffers from some limitations for quantitative work (how much is present). It also is not useful in liquid feed applications and thorough and careful mixing of the particle with the feed additive is required for these products. Particle size of the iron particle or coded tracer also will play a role in this distribution. While it is theoretically possible to quantify the animal feed in these systems, in reality it works much better as a qualitative method.

The reasons for a need to be able to detect quantitatively the amount of feed additive added to an animal feed are several. Amongst them are complaints that feed additive manufacturers hear from time to time that farmers/users are not getting the results that the additive or supplement manufacturer claims. In many instances close investigation will reveal that the problem is not at all with the feed additive but the problem is in failure of the farmer/user to properly follow the manufacturer's instructions. The most common occurrences are that incorrect levels, not within the recommended manufacturer levels are being used. That is to say, either too much or too little is used, or the additive is not homogeneously admixed with the feed. Too much is undesirable because it wastes product. Too little is undesirable because the expected and looked for results will not be achieved. Proper distribution is desirable because animals feeding for example at one end of a feed bunk may get none and animals feeding at the other end may get it all, dosing themselves at way too high a level. Its therefore not only using correct levels that is important but it is important that they be properly admixed with the feed mixture to provide a homogeneous dry mix.

Troubleshooting these farmer/user problems can be difficult. To aid and assist, the systems previously discussed in the Background of Invention, have been employed from time to time. Their limits have been explained. Other techniques have in the past also been tried.

An analysis of chemical compounds in samples exploiting the fact that some compounds (fluorophors) exhibit fluorescence when excitated with light (fluorescence analysis) are well known to the art. While fluorescent analysis has its advantages in being sensitive and accurate in well-defined samples, it also has serious drawbacks. For example in complex samples the fluorescence of the fluorophor is often altered by other compounds present in the environment surrounding the fluorophor (known as quenching) making it difficult to use a fluorescence analysis quantitatively on complex and/or poorly defined samples. This applies especially in heterogeneous or solid phase samples where scattering of the light also has to be accounted for. See for example U.S. 2002/0127586 A1 published Sep. 12, 2002 to fluorescence detection of biologically active compounds such as enzymes by illuminating the granular enzymatic component as a granular fluorescent detector or marker. Another example is reported in Journal of Animal Science, 2001, 79:2731-2735, Technical Note: Method for Detecting Liquid Enzyme Additives Added to Animal Feed. This involves a presence/absence detection of liquid enzymes using a fluorescent tracer.

In all of the above detection systems here referenced, whether using micro tracers, magnetic tracers, coded tracers, or even fluorescent tracers, the problem is that the system does presence/absence detection (qualitative detection) and does not allow quantitative detection to determine whether or not the user/farmers are complying with manufacturer use instructions. Therefore, troubleshooting customer complaints is difficult.

For one particular class of mixed additives quantitative detection is especially important. This class of additives can generally be referred to as mineral additives that use trace elements complexed with alpha amino acids that are typical essential amino acids, such as lysine and methionine. Here knowing both presence, and amount is important.

For proper health, trace elements must be provided in the diet both in sufficient quantities and in a form which can be utilized by the animal. The degree to which an ingested substances is absorbed in a form that is utilizable is defined as “bioavailability”. The bioavailability of nutrients for animals is summarized in a comprehensive monograph edited by Ammerman, Baker and Lewis published in 1995.

Many commercial products have been developed as additives to enhance the bioavailability of the trace elements from animal feed. The beneficial effects of these products are attributed to the association of the metal with an organic molecule, usually called a ligand. This association or bonding results in the increased availability of the metal for utilization by animals, i.e. increased bioavailability. The increased bioavailability of the trace elements in these products is a result of increased solubility, greater stability in the gut, enhanced absorption into circulation and/or improved metabolic utilization.

Different types of products that contain a trace element associated with an organic ligand are commercially available. These can be classified in different groups based on the nature of the ligand used in manufacturing the product. In one class of products, amino acids are used as the ligands that form complexes or chelates with the metal. Examples of these products are described in U.S. Pat. Nos. 3,941,818, 3,950,372, 4,067,994, 4,900,651, 4,948,594, 4,956,188, 5,061,815, 5,278,329, 5,583,243, 4,863,898 and 6,166,071. A second group of feed additives include the metal salts of short chain carboxylic acids such as propionic acid (see U.S. Pat. Nos. 5,591,878, 5,707,679, 5,795,615 and 5,846,581). A third group of trace element additives is classified by the American Feed Control Officials as Metal Proteinate and defined as “the product resulting from the chelation of a soluble salt with amino acids and/or partially hydrolyzed protein”. Examples of these products are described in U.S. Pat. Nos. 3,440,054, 3,463,858, 3,775,132, 3,969,540, 4,020,158, 4,076,803, 4,103,003, 4,172,072 and 5,698,724.

A primary object of the present invention is to provide methods of quantitative detection of feed additives, particularly trace element, amino acid complex feed additives.

Another object of the invention is to manufacture feed additive premixes that when added to feed contain a fluorescent detector that allows quantitative detection when they are mixed with animal feed.

The method and means of accomplishing the above objectives as well as others will become apparent from the detailed description of the invention which follows hereinafter.

### BRIEF SUMMARY OF THE INVENTION

Compositions for detecting and measuring quantitatively amounts of admixed feed additives added to animal feed mixes, using soluble tracer fluorophors, mixed with a liquid form of the additive during the manufacturing process.

### DETAILED DESCRIPTION OF A PREFERRED EMBODIMENT

The present invention relates, to a method for quantitative detection of solubilized feed additives admixed with common livestock feeds. The livestock feeds to which the feed additives may be added will vary depending upon the feed animal, but generally it includes cattle, hogs, poultry, sheep, etc. Typical feeds for these vary considerably depending upon the animal but include minerals, mashes, silage, etc. Depending upon the feed, usually the feed additive is added dry and stirred in or spread over as a top dressing. The additive method and amount used by various manufacturers will vary depending upon the supplement manufacturer, and the supplement being fed. For example, for Zinpro Corporation for its feed additives that comprise trace element amino acid complexes, typical feed level recommendations are at varied levels depending upon whether the animal is swine, poultry, beef cattle, dairy cattle, sheep or horses (but usually varies from for example one pound per ton of feed ration for swine and poultry to 2.5 grams/day for beef and dairy cattle. These products are sold under the trademark Zinpro, and other trademarks such as Availa. Trace metals can be zinc, copper, magnesium, cobalt, or in the case of Availa, a mixture of amino acids; or they can be things such as zinc methionine complexes or copper lysine complexes. In accordance with the present invention, the tracer is added to the solubilized (water) feed additive and it is in a liquid state during the manufacturing process. It is important that the tracer be a water soluble tracer. Water soluble fluorescein tracers include the following: Fluorescein (Uranin), Rhodamine WT, Rhodamine B, Rhodamine 6G, Acid Red, Acid Yellow, Eosin, Tropinal CBS-X, Pyranine, Phorwite BBH, Tinopal 5BM GX, Diphenyl Brilliant Flavine 7GFF, Lissamine Flavine FF, Sulpho Rhodamine G.

The amount of the water soluble fluorescein tracer added will vary but generally can be within the range of from 0.0005% to 0.05%, preferably 0.0025% to 0.01%, and most preferred at 0.01%. Any one of the available water soluble fluorescein tracers above mentioned can be used. The key to the amount is an amount sufficient to allow accurate quantitative measurement and detection.

The optical properties of the fluorescent marker compound may be known and to optimize the excitation of a known fluorescent marker it is preferred to select a light source delivering a substantial portion of light of wavelengths suitable for excitating the fluorescent marker. If it is desired (which it may be when using a known fluorescent marker) to avoid or limit excitation and emission from compounds other than the fluorescent marker, which may interfere with the analysis, one can filter the beam of light, so that only light of selected wavelengths illuminates. This may be done with one or more beam splitters and one or more band pass filters, such as high and/or low band pass filters, or grate monochromaters allowing only light with specific wavelengths to pass. These features are normally integrated in commercially available fluorescence analyzers, e.g. from Perkin Elmer, USA. It is known to the skilled person that band pass filters and monochromaters will allow passage of light having wavelengths within a narrow ranges, normally within a few nm, such as 0.5-10 nm. Thus the term monochromatic light is to be understood as light having wavelengths within the narrow range determined by the band pass filter or the grate monochromater.

After admixing of the water soluble fluorescein tracer marker homogeneously with the animal feed supplement, the manufacturing process can continue in a conventional manner to drying, which may be oven drying, spray drying, any other conventional means of feed additive drying to go from the liquid state to the dry powder state. It may then be mixed with other conventional dry food ration supplements and sold as a feed additive.

In the detection process, if for example one is troubleshooting a complaint from a farmer/user for these products, samples of the product can be obtained, solubilized and the homogeneous relationship between the fluorescein tracer and additive allows for detection at a quantitative level by conventional fluorescein analysis, using well known detecting techniques for fluorescent markers. Since the description is well known, it need not be repeated herein.

The following examples are offered to further illustrate but not limit the process of the invention.

The first example of a feed additive is a known available product from Zinpro Corporation of Eden Prairie, Minn. similar to Availa 4 but without the cobalt. It comprises a mixture of zinc, manganese copper amino acid complexes. Other examples include zinc and methionine complex and a copper lysine complexes.

### EXAMPLES

The HPLC conditions for the analysis in the examples are described as follows:

### Equipment:

Shimadzu SCL 10A VP System Controller
Shimadzu LC-10AD VP Pumps (2)
Shimadzu SPD-10V VP UV-Vis Detector
Shimadzu SIL-10Ai Auto injector
Shimadzu CTO-20AC Column Oven
Shimadzu RF-10A XL Fluorescence Detector

### HPLC Run Conditions (Uranine):

Column: ACE 3μ C18 150×4.6 mm
Combined Mobile Phase:
   1. Mobile Phase A: 50 mM Phosphate Buffer prepped at pH 7.1
   2. Mobile Phase B: MeOH
   3. Ratio: MP-A/MP-B (60/40)
Oven: 35° C.
Flow Rate: 0.8 mL/min
Injection Volume: 5 μL
λₑₓ=460; λₑₘₘ=515
Isocratic conditions
Run time=8.0 minutes
Diluent is 200 mM phosphate buffer/DI water (60/40)

### Example 1

### Combination Metal Amino Acid Complexes

A reactor vessel used in the hydrolysis of feathermeal protein to amino acids was a DeDiedrich, Inc model CTJ-32-100 glass lined steel clamp top jacket reactor vessel. Approximately 176 pounds of water and 410 pounds of 31.4% hydrochloric acid were added to the vessel. To this acid solution was added 240 pounds of feather meal. The feather meal was hydrolyzed by heating the mixture to 140° C. under pressure and holding it there for 1 hr. The vessel was then allowed to cool and vent. At this stage, while continuing to provide agitation, 67 pounds of zinc oxide (80% Zn) was added and allowed to dissolve. Next, 25 pounds of copper oxide (75% Cu) was added and allowed to dissolve. Next, 38 pounds of manganese oxide (77% Mn) was added and allowed to dissolve. Finally, 84 pounds of 50% w/w sodium hydroxide was added and the final pH was about 4.1. This combination metal amino acid complex solution was determined to be 49% solids.

### Example 2

The liquid hydrolyzate mixture (441.3 gr) prepared in Example 1 was added to a 1000 mL glass beakers and stirred. To this solution was added uranine tracer (Fischer, 37.6 mg) and stirring continued for 0.5 hrs. To this was added 158.8 gr of corn cob carrier to make a paste that was dried in an oven at 70° C. in drying pans. The material was turned over periodically with a spatula during the drying process. Once dried it was ground with a mortar and pestle and sieved through an 850 micron sieve to remove any large particles. This product contains 0.01% by weight uranine.

### Example 3

To a sample of corn (143 gr) was added 0.07 wt % of the product prepared in example 2. This mixture was then extracted and analyzed via HPLC to determine the amount of uranine tracer in the feed sample. The extraction conditions were to mix 100 gr of feed with 100 mL of a 60/40 mixture of 200 mM phosphate buffer/DI water which is then stirred for 25 minutes. The mixture is then filtered and the solution analyzed by HPLC by the conditions previously given. The results were then referenced back to the level in the actual product to determine the amount of product in the feed. The theoretical amount of product in the feed was 0.07% and the amount calculated based on the HPLC of the uranine tracer was 0.068% or 97.6% of the actual amount.

### Example 4

A sample of zinc methionine complex was prepared by dissolving ZnSO4 (135.7 gr) and methionine (112.9 gr) in solution with heating to 70° C. in 300 mL water. To this was added 24.8 mg of Rhodamine B (Sigma) as the tracer. The mixture was dried down to yield a product with 0.01% tracer homogeneous with the product. Once dried it was ground with a mortar and pestle and sieved through an 850 micron sieve to remove any large particles.

### Example 5

The zinc methionine complex prepared with Rhodamine B tracer in example 4 was added at a level of 0.07% to corn. The feed mixture was then extracted with a mixture of water and phosphate buffer (60% 200 mM Phosphate Buffer and 40% DI water) and analyzed by HPLC. The HPLC conditions were identical to example 3 except the solvent system was run at a ratio of MP-A/MP-B (30/70) and the fluorescence excitation was 540 nm and the detector for emission was set at 625 nm. The analysis determined the amount product in the corn to be 0.067% or 97% of the actual amount.

### Example 6

A sample of a copper lysine complex was prepared by dissolving CuSO4 (162.4 gr) and Lysine HCl (241.2 gr) in a solution with 350 mL H₂O. To this mixture was added NaOH (36.1 gr) and the entire mixture heated to 70° C. for 45 minutes. 3-Hydroxypyrene 5,8,10-Tri Sulfonic acid (HPSA, Aldrich) (39.2 mg) was added and the mixture was stirred for 1 hr and then concentrated by rotary evaporation. The product was then transferred to a drying pan and further dried in at oven at 70° C. Once dried it was ground with a mortar and pestle and sieved through an 850 micron sieve to remove any large particles. This product will then be utilized in Example 7.

### Example 7

The copper lysine complex prepared in example 6 was added at a level of 0.07% to corn. The feed mixture was then extracted with a mixture of water and phosphate buffer (60% 200 mM Phosphate Buffer and 40% DI water) and analyzed by HPLC. The extraction procedure was to add 100 gr of the corn mixture to 100 mL of diluents as described in previous examples. The solvent was then filtered and the solution analyzed by HPLC. The HPLC conditions were identical to example 3 except the solvent system was run at a ratio of MP-A/MP-B (85/15) and the fluorescence excitation was 403 nm and the detector for emission was set at 513 nm. The analysis determined the amount product in the corn to be 0.068% or 98% of the actual amount.

### Example 8

The liquid hydrolyzate mixture (408.2 gr) prepared in Example 1 was added to a 1000 mL glass beakers and stirred. To this solution was added Rhodamine 6G tracer (Sigma, 34.7 mg) and stirring continued for 0.5 hrs. To this was added 146.8 gr of corn cob carrier to make a paste that was dried in an oven at 70° C. in drying pans. The material was turned over periodically with a spatula during the drying process. Once dried it was ground with a mortar and pestle and sieved through an 850 micron sieve to remove any large particles. This product contains 0.01% by weight Rhodamine 6G.

### Example 9

To a sample of corn (143 gr) was added 0.07% of the product prepared in example 1. This mixture was then extracted as previously described and analyzed via HPLC to determine the amount of Rhodamine 6G tracer in the feed sample. This was then referenced back to the level in the actual product to determine the amount of product in the feed. The HPLC conditions were identical to example 3 except the solvent system was run at a ratio of MP-A/Acetonitrile (50/50) and the fluorescence excitation was 526 nm and the detector for emission was set at 555 nm. The analysis determined the amount product in the corn to be 0.065% or 93% of the actual amount.

From the above examples, it can be seen that the tracer system of the present invention is a reliable and accurate way of determining the presence, and the amount of product in feed samples.

What is claimed is:

1. A quantitative detection method for levels of soluble feed additives mixed with animal feed, comprising:
adding a water soluble fluorescent tracer to a solubilized feed additive during the feed additive preparation process;
mixing until homogenous the solubilized feed additive and the water soluble fluorescent tracer to provide a solubilized mix;
drying the solubilized mix to provide a traceable commercial feed additive;
mixing the traceable commercial feed additive with animal feed;
thereafter detecting quantitatively the amount of soluble feed additive by detecting and measuring the water soluble fluorescent tracer.

2. The process of claim 1 wherein the feed additive is a trace metal feed additive, with trace metal selecting from the group consisting of zinc, copper, magnesium, cobalt, iron, selenium and chromium, and mixtures hereof.

3. The process of claim 2 wherein the animal feed is selected from the group consisting of minerals, mashes and silage.

4. The process of claim 2 wherein the water soluble fluorescent tracer is selected from the group consisting of: Fluorescein (Uranin), Rhodamine WT, Rhodamine B, Rhodamine 6G, Acid Red, Acid Yellow, Eosin, Tropinal CBS-X, Pyranine, Phorwite BBH, Tinopal 5BM GX, Diphenyl Brilliant Flavine 7GFF, Lissamine Flavine FF, Sulpho Rhodamine G.

5. The process of claim 4 wherein the amount of the water soluble fluorescent tracer is from 0.0005% by weight to 0.05% by weight.

6. The process of claim 5 wherein the amount of water soluble fluorescent tracer is from 0.0025% by weight to 0.01% by weight.

7. A composition of matter in the form of a homogeneous feed premix additive comprised of a soluble feed additive that has been previously homogeneously admixed with a water soluble fluorescent tracer and then dried.

8. The composition of claim 7 wherein the feed additive is a trace metal feed additive, the trace metal being selected from the group consisting of zinc, copper, magnesium, cobalt, iron, selenium and chromium, and mixtures hereof.

9. The composition of claim 7 wherein the animal feed is selected from the group consisting of minerals, mashes and silage.

10. The composition of claim 7 wherein the water soluble fluorescent tracer is selected from the group consisting of: Fluorescein (Uranin), Rhodamine WT, Rhodamine B, Rhodamine 6G, Acid Red, Acid Yellow, Eosin, Tropinal CBS-X, Pyranine, Phorwite BBH, Tinopal 5BM GX, Diphenyl Brilliant Flavine 7GFF, Lissamine Flavine FF, Sulpho Rhodamine G.

11. The composition of claim 10 wherein the amount of the water soluble fluorescent tracer is from 0.0005% by weight to 0.05% by weight.

12. The composition of claim 11 wherein the amount of water soluble fluorescent tracer is from 0.0025% by weight to 0.01% by weight.
